(19) United States
(12) Patent Application Publication
(10) Pub. No.: US 2016/0158231 A1
(43) Pub. Date: Jun. 9, 2016

(54) PYRIMIDINE HYDROXY AMIDE COMPOUNDS FOR TREATING PERIPHERAL NEUROPATHY

(71) Applicants: VIB VZW, Gent (BE); ACETYLON PHARMACEUTICALS, INC., Boston, MA (US); LIFE SCIENCES RESEARCH PARTNERS VZW, Leuven (BE); Katholieke Universiteit Leuven, K.U. Leuven R&D, Leuven (BE)

(72) Inventors: Matthew Blaire Jarpe, Quincy, MA (US); Ludo Van Den Bosch, Arendonk (BE); Veronick Benoy, Tienen (BE); Lawrence Van Helleputte, Bierbeek (BE)

(21) Appl. No.: 14/959,317

(22) Filed: Dec. 4, 2015

Related U.S. Application Data
(60) Provisional application No. 62/088,173, filed on Dec. 5, 2014.

(51) Int. Cl. A61K 31/505

(52) U.S. Cl. CPC A61K 31/505

(57) ABSTRACT

The invention relates to HDAC inhibitors for the treatment of peripheral neuropathy in a subject in need thereof. Also provided herein are methods for treating peripheral neuropathy in a subject in need thereof comprising administering to the subject a therapeutically effective amount of an HDAC inhibitor.

### CROSS-REFERENCE TO RELATED APPLICATIONS

This application claims priority to U.S. Provisional Application Ser. No. 62/088,173, filed Dec. 5, 2014, which is incorporated herein by reference in its entirety.

### BACKGROUND

Histone deacetylase (HDAC) enzymes represent attractive therapeutic targets in the treatment of peripheral neuropathy.

### SUMMARY

Provided herein are pharmaceutical compounds for the treatment of peripheral neuropathies in a subject in need thereof. Also provided herein are methods for treating a peripheral neuropathy in a subject in need thereof.

In one aspect, provided herein are methods of treating a peripheral neuropathy in a subject in need thereof comprising administering to the subject a therapeutically effective amount of a compound of Formula (I):

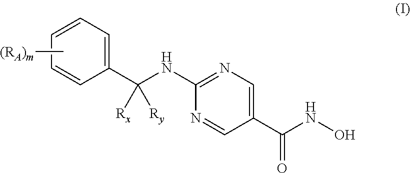

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH or haloalkyl; and
m is 0, 1, or 2.

In an embodiment, the peripheral neuropathy is Charcot-Marie Tooth Disease.

In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy. In an embodiment, the chemotherapy-induced peripheral neuropathy is taxol-induced peripheral neuropathy.

In another aspect, provided herein are methods of treating or preventing a peripheral neuropathy in a subject in need thereof comprising administering to the subject a therapeutically effective amount of the compound:

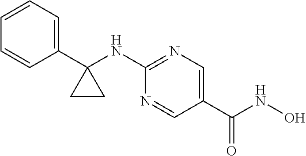 [SMILES: O=C(NO)c1cnc(NC2(c3ccccc3)CC2)nc1]

or a pharmaceutical composition comprising the compound, to thereby treat or prevent the peripheral neuropathy. In one embodiment, the peripheral neuropathy is Charcot-Marie Tooth Disease. In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy.

In yet another aspect, provided herein are methods of treating or preventing a peripheral neuropathy in a subject in need thereof comprising administering to the subject a therapeutically effective amount of the compound:

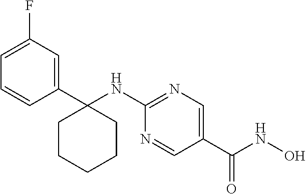 [SMILES: O=C(NO)c1cnc(NC2(c3cccc(F)c3)CCCCC2)nc1]

or a pharmaceutical composition comprising the compound, to thereby treat or prevent the peripheral neuropathy. In one embodiment, the peripheral neuropathy is Charcot-Marie Tooth Disease. In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy. In an embodiment of any of the methods of the invention, the subject is a human.

### BRIEF DESCRIPTION OF THE FIGURES

FIG. 1 shows the mean response to Von Frey test (g) in the study of vincristine-induced neuropathy in rat described in Example 6.

FIG. 2 shows the mean response to Von Frey test (g) in the study of taxol-induced neuropathy in rat described in Example 7.

FIG. 3A. 1 μM of Compound A and Compound B increase the levels of acetylated α-tubulin in N2a cells to similar levels as 1 μM TSA. Only TSA increases the acetylation of H3. α-tubulin and histone 4 (H4) were used as a loading control. (See Example 8)

FIG. 3B. Ratios between the intensity of acetylated α-tubulin and α-tubulin, normalized to TSA, show that Compound A and Compound B significantly increase acetylated α-tubulin compared to DMSO treated cells. *** p<0.0001, N=3. (See Example 8)

FIG. 3C. Ratios between the intensity of acetylated H3 and H4, normalized to DMSO, show that Compound A and Compound B do not change the acetylation of H3. (See Example 8)

FIG. 3D. TSA and Compound B already increase the acetylation of α-tubulin at lower concentrations starting from 10 nM while Compound A is less potent. α-tubulin were used as a loading control. (See Example 8)

FIG. 3E. Dose-response curves based on the ratios between the intensity of acetylated α-tubulin and α-tubulin on WB show that TSA and Compound B are more potent HDAC6 inhibitors than Compound A. Two-way ANOVA, *p<0.05. (See Example 8)

FIG. 3F. Immunofluorescent staining of 1 μM-treated N2a cells show that all compounds increase the acetylation of a-tubulin (red) while only TSA affects the histone acetylation in the nucleus (green). (See Example 8)

FIGS. 4A-C. Compound A and Compound B are able to increase the number of moving and total mitochondria in the neurites in mutant HSPB1 DRG neurons, while the amount of stationary mitochondria remains unchanged. (See Example 9)

FIG. 4D. Kymographs constructed from DMSO, Compound A and Compound B treated DRG neurons show stationary mitochondria as vertical lines and to the right or left deflecting lines as respectively anterograde or retrograde moving mitochondria. (See Example 9)

FIG. 4E. Both compounds also increased the levels of acetylated α-tubulin in the neurites of the Compound A or Compound B treated DRG neurons. (See Example 9)

FIG. 5A. Compound A and Compound B (all 3 mg/kg), Tubastatin A and Compound B can restore the amplitude of the compound muscle action potential (CMAP) of mutant HSPB1 mice. (See Example 10)

FIG. 5B shows the effect of daily IP injection of Tubastatin A, Compound A or Compound B on the SNAP amplitudes. (See Example 10)

FIG. 5C shows the motor performance on the rotarod. (See Example 10)

FIG. 5D shows the innervation of the gastrocnemius muscle as determined by counting the number of innervated neuromuscular junctions (NMJs). One-way ANOVA, *p<0.05, n=3-5. (See Example 10)

FIG. 6A. Vincristine-induced a sensory neuropathy in mice. Female C57B16/J mice received a daily i.p. injection with either vehicle or vincristine. Vincristine (150 μg/kg) induced a peripheral neuropathy, hallmarked by a reduction in the Sensory Nerve Action Potential (SNAP) amplitudes (−35.4%), while the latencies were unaffected. This neuropathy was already present after 7 days of vincristine treatment and was not further increased after 14 days of treatment. (See Example 11)

FIG. 6B. After 7 days of vincristine treatment, a subset of animals was further co-treated with vincristine and a specific HDAC6-inhibitor, Tubastatin A (i.p.; 25 mg/kg/day). At day 14, the vincristine-induced reduction in SNAP amplitudes was completely rescued in the treatment group that also received Tubastatin A. (See Example 11)

FIG. 6C. Similar to FIG. 6B, mice were first treated with vincristine and from day 7 on, 3 groups were co-treated with vincristine and either 3, 10 or 25 mg/kg (ip) Compound A, a HDAC6 inhibitor. ****p<0.001, VCR: Vincristine. (See Example 11).

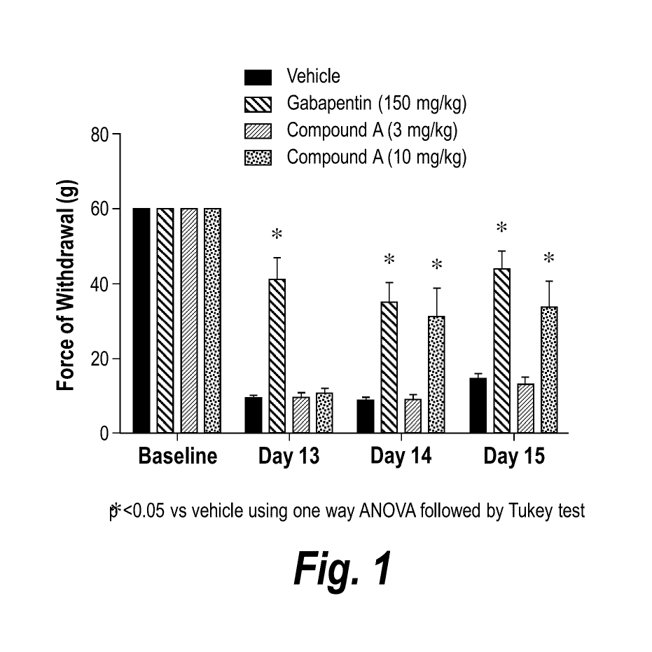

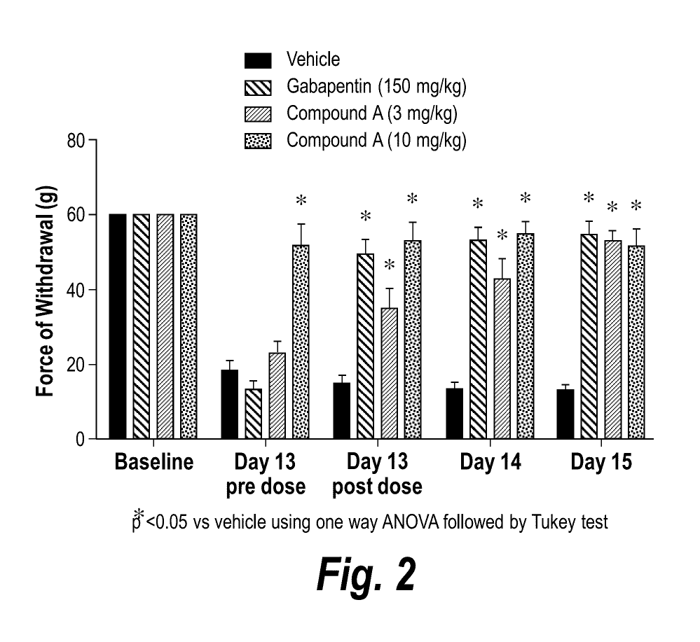

### DETAILED DESCRIPTION

### Definitions

Listed below are definitions of various terms used to describe this invention. These definitions apply to the terms as they are used throughout this specification and claims, unless otherwise limited in specific instances, either individually or as part of a larger group.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The number of carbon atoms in an alkyl substituent can be indicated by the prefix “C_{x-y}”, where x is the minimum and y is the maximum number of carbon atoms in the substituent. Likewise, a Cₓ chain means an alkyl chain containing x carbon atoms.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The term “alkoxy” refers to an —O-alkyl moiety.

The terms “halo” and “halogen” refer to an atom selected from fluorine, chlorine, bromine and iodine. The term “haloalkyl” refers to an alkyl group with one more instances of halo substitution, e.g., —CF₃.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “inhibitor” is synonymous with the term antagonist.

The term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Additionally, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

Combinations of substituents and variables envisioned by this invention are only those that result in the formation of stable compounds. The term “stable” refers to compounds which possess stability sufficient to allow manufacture and which maintains the integrity of the compound for a sufficient period of time to be useful for the purposes detailed herein (e.g., therapeutic or prophylactic administration to a subject).

The term “subject” refers to a mammal. A subject therefore refers to, for example, dogs, cats, horses, cows, pigs, guinea pigs, and the like. Preferably the subject is a human. When the subject is a human, the subject may be referred to herein as a patient.

The terms “treating,” “treatment” and the like are used herein to mean obtaining a desired pharmacologic and/or physiologic effect that at least alleviates or abates a disease and/or its attendant symptoms. “Treating” also covers any treatment of a disease in a mammal, and includes: (a) preventing a disease from occurring in a subject that may be predisposed to that disease, but has not yet been diagnosed as having it; (b) inhibiting a disease, i.e., arresting its development; or (c) relieving or ameliorating the disease, e.g., cause regression of the disease. As used herein, to “treat” includes systemic amelioration of the symptoms associated with the pathology and/or a delay in onset of symptoms. Clinical and sub-clinical evidence of “treatment” will vary with the pathology, the individual and the treatment.

The terms “prevent,” “preventing,” and “prevention” refer to administration of the compound of the invention, or a pharmaceutical composition in order to prevent a disease, condition, or disorder in an individual who may be predisposed or likely to exhibit said disease, condition, or disorder, but does not yet experience or exhibit the pathology or symptomatology of the disease, condition, or disorder.

### Histone Deactylase (HDAC) Inhibitors

The HDAC6-selective inhibitor provided herein is a compound of Formula (I):

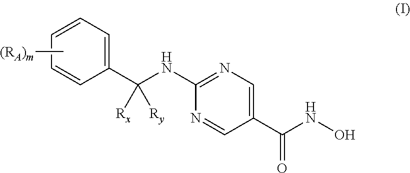

or a pharmaceutically acceptable salt thereof,

wherein,

Rₓ and R_{y} together with the carbon to which each is attached, form a cyclopropyl, cyclobutyl, cyclopentyl, cyclohexyl, cycloheptyl, or cyclooctyl;

each R_{A} is independently C₁₋₆-alkyl, C₁₋₆-alkoxy, halo, OH, or haloalkyl; and

m is 0, 1, or 2.

In an embodiment of the compound s of Formula (I), m is 0 or 1.

Representative compounds of Formula (I) include, but are not limited to, compounds selected from Table 1:

TABLE 1

| 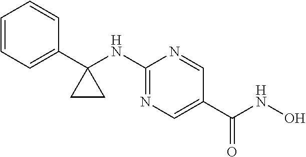 [SMILES: O=C(NO)c1cnc(NC2(c3ccccc3)CC2)nc1] | 73, Compound A |
|---|---|
| 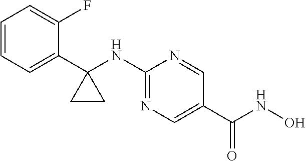 [SMILES: O=C(NO)c1cnc(NC2(c3ccccc3F)CC2)nc1] | 82 |
| 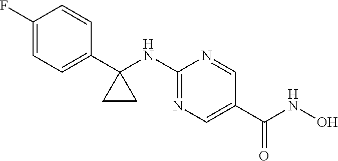 [SMILES: O=C(NO)c1cnc(NC2(c3ccc(F)cc3)CC2)nc1] | 81 |
| 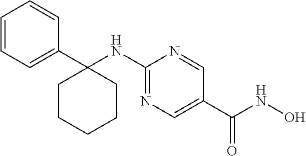 [SMILES: O=C(NO)c1cnc(NC2(c3ccccc3)CCCCC2)nc1] | 84 |
| 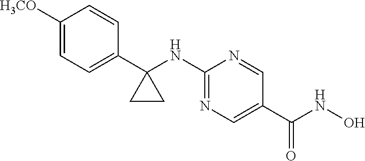 [SMILES: COc1ccc(C2(Nc3ncc(C(=O)NO)cn3)CC2)cc1] | 97 |
| 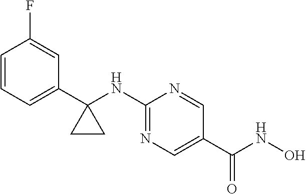 [SMILES: O=C(NO)c1cnc(NC2(c3cccc(F)c3)CC2)nc1] | 87 |
| 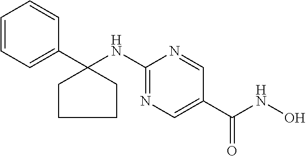 [SMILES: O=C(NO)c1cnc(NC2(c3ccccc3)CCCC2)nc1] | 88 |
| 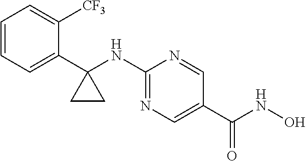 [SMILES: O=C(NO)c1cnc(NC2(c3ccccc3C(F)(F)F)CC2)nc1] | 91 |
| 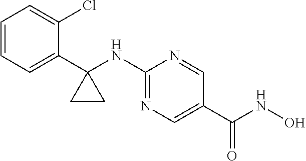 [SMILES: O=C(NO)c1cnc(NC2(c3ccccc3Cl)CC2)nc1] | 90 |
| 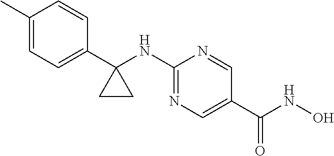 [SMILES: Cc1ccc(C2(Nc3ncc(C(=O)NO)cn3)CC2)cc1] | 93 |
| 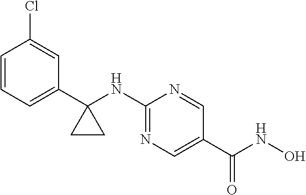 [SMILES: O=C(NO)c1cnc(NC2(c3cccc(Cl)c3)CC2)nc1] | 94 |
| 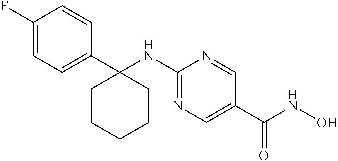 [SMILES: O=C(NO)c1cnc(NC2(c3ccc(F)cc3)CCCCC2)nc1] | 100 |
| 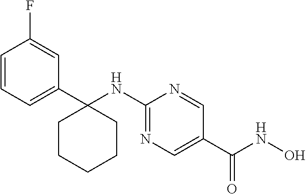 [SMILES: O=C(NO)c1cnc(NC2(c3cccc(F)c3)CCCCC2)nc1] | 101, Compound B |
| 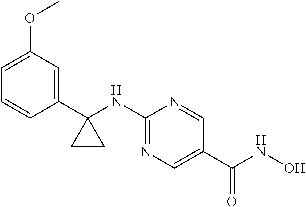 [SMILES: COc1cccc(C2(Nc3ncc(C(=O)NO)cn3)CC2)c1] | 120 |
| 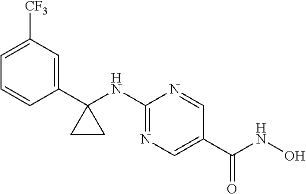 [SMILES: O=C(NO)c1cnc(NC2(c3cccc(C(F)(F)F)c3)CC2)nc1] | 121 |
| 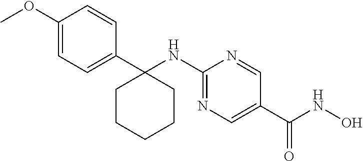 [SMILES: COc1ccc(C2(Nc3ncc(C(=O)NO)cn3)CCCCC2)cc1] | 122 |
| 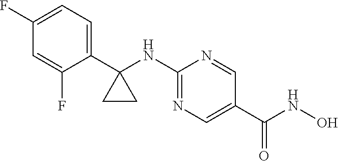 [SMILES: O=C(NO)c1cnc(NC2(c3ccc(F)cc3F)CC2)nc1] | 123 |
| 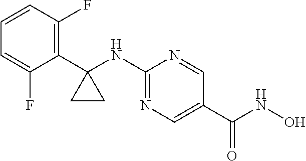 [SMILES: O=C(NO)c1cnc(NC2(c3c(F)cccc3F)CC2)nc1] | 124 |
| 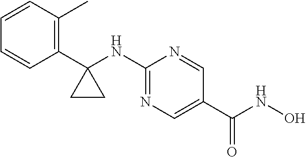 [SMILES: Cc1ccccc1C1(Nc2ncc(C(=O)NO)cn2)CC1] | 127 |
| 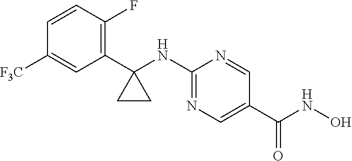 [SMILES: O=C(NO)c1cnc(NC2(c3cc(C(F)(F)F)ccc3F)CC2)nc1] | 126 |
| 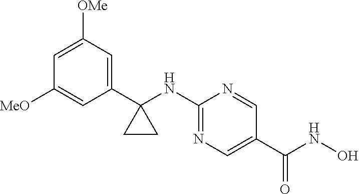 [SMILES: COc1cc(OC)cc(C2(Nc3ncc(C(=O)NO)cn3)CC2)c1] | 129 |
| 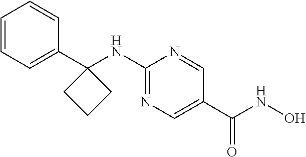 [SMILES: O=C(NO)c1cnc(NC2(c3ccccc3)CCC2)nc1] | 130 |
| 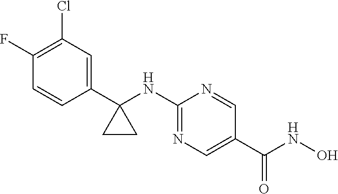 [SMILES: O=C(NO)c1cnc(NC2(c3ccc(F)c(Cl)c3)CC2)nc1] | 131 |
| 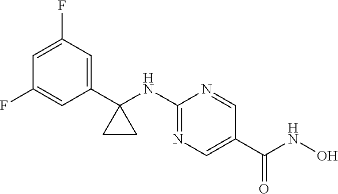 [SMILES: O=C(NO)c1cnc(NC2(c3cc(F)cc(F)c3)CC2)nc1] | 132 |
| 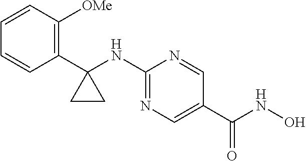 [SMILES: COc1ccccc1C1(Nc2ncc(C(=O)NO)cn2)CC1] | 133 |
| 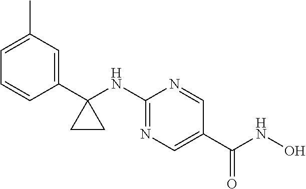 [SMILES: Cc1cccc(C2(Nc3ncc(C(=O)NO)cn3)CC2)c1] | 134 |
| 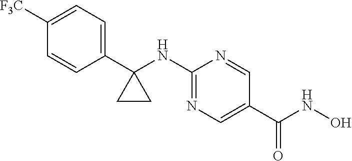 [SMILES: O=C(NO)c1cnc(NC2(c3ccc(C(F)(F)F)cc3)CC2)nc1] | 135 |
| 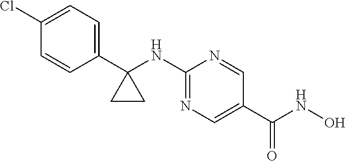 [SMILES: O=C(NO)c1cnc(NC2(c3ccc(Cl)cc3)CC2)nc1] | 136 |
| 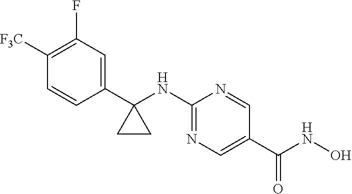 [SMILES: O=C(NO)c1cnc(NC2(c3ccc(C(F)(F)F)c(F)c3)CC2)nc1] | 137 |
| 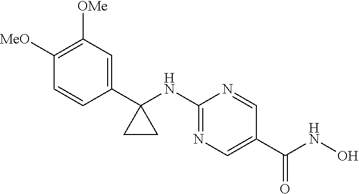 [SMILES: COc1ccc(C2(Nc3ncc(C(=O)NO)cn3)CC2)cc1OC] | 138 |
| 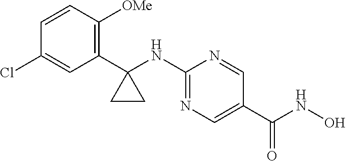 [SMILES: COc1ccc(Cl)cc1C1(Nc2ncc(C(=O)NO)cn2)CC1] | 139 |
| 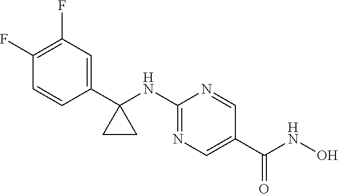 [SMILES: O=C(NO)c1cnc(NC2(c3ccc(F)c(F)c3)CC2)nc1] | 140 |
| 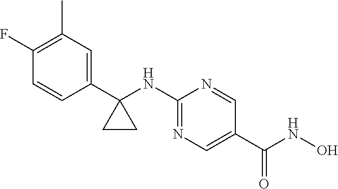 [SMILES: Cc1cc(C2(Nc3ncc(C(=O)NO)cn3)CC2)ccc1F] | 142 |
| 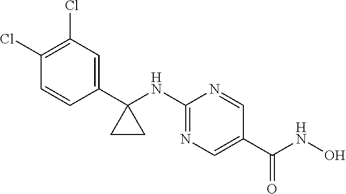 [SMILES: O=C(NO)c1cnc(NC2(c3ccc(Cl)c(Cl)c3)CC2)nc1] | 141 |
| 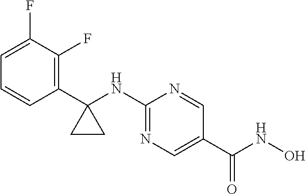 [SMILES: O=C(NO)c1cnc(NC2(c3cccc(F)c3F)CC2)nc1] | 143 |
| 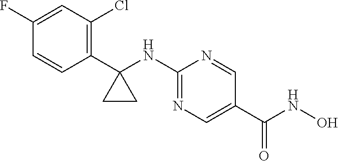 [SMILES: O=C(NO)c1cnc(NC2(c3ccc(F)cc3Cl)CC2)nc1] | 144 |
| 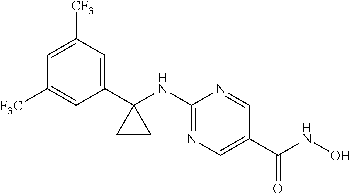 [SMILES: O=C(NO)c1cnc(NC2(c3cc(C(F)(F)F)cc(C(F)(F)F)c3)CC2)nc1] | 145 |
| 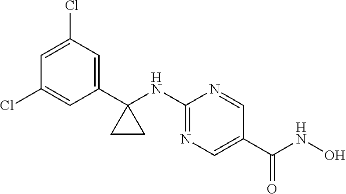 [SMILES: O=C(NO)c1cnc(NC2(c3cc(Cl)cc(Cl)c3)CC2)nc1] | 146 |
| 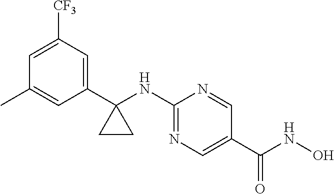 [SMILES: Cc1cc(C(F)(F)F)cc(C2(Nc3ncc(C(=O)NO)cn3)CC2)c1] | 147 |
| 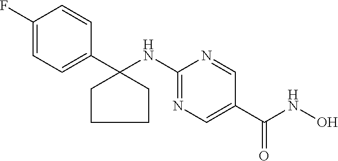 [SMILES: O=C(NO)c1cnc(NC2(c3ccc(F)cc3)CCCC2)nc1] | 148 |
| 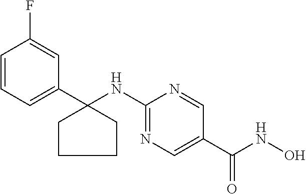 [SMILES: O=C(NO)c1cnc(NC2(c3cccc(F)c3)CCCC2)nc1] | 151 |
| 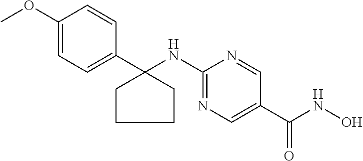 [SMILES: COc1ccc(C2(Nc3ncc(C(=O)NO)cn3)CCCC2)cc1] | 152 |
| 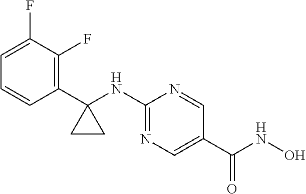 [SMILES: O=C(NO)c1cnc(NC2(c3cccc(F)c3F)CC2)nc1] | 143 |
| 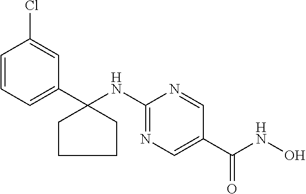 [SMILES: O=C(NO)c1cnc(NC2(c3cccc(Cl)c3)CCCC2)nc1] | 153 |

and pharmaceutically acceptable salts thereof.

The compounds of Formula (I) may be further selected from Table 2.

TABLE 2

| | |
|---|---|
| 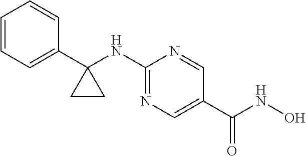 [SMILES: O=C(NO)c1cnc(NC2(c3ccccc3)CC2)nc1] | 73 |
| 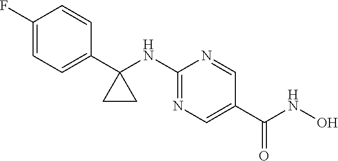 [SMILES: O=C(NO)c1cnc(NC2(c3ccc(F)cc3)CC2)nc1] | 81 |
| 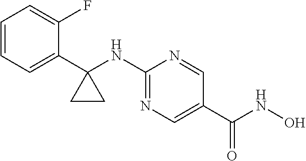 [SMILES: O=C(NO)c1cnc(NC2(c3ccccc3F)CC2)nc1] | 82 |
| 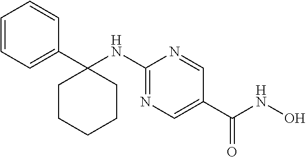 [SMILES: O=C(NO)c1cnc(NC2(c3ccccc3)CCCCC2)nc1] | 84 |
| 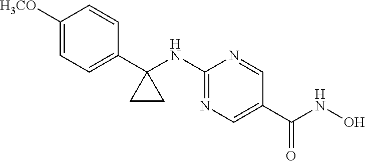 [SMILES: COc1ccc(C2(Nc3ncc(C(=O)NO)cn3)CC2)cc1] | 97 |
| 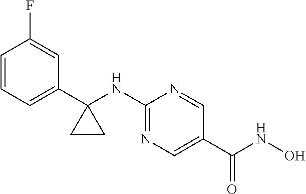 [SMILES: O=C(NO)c1cnc(NC2(c3cccc(F)c3)CC2)nc1] | 87 |
| 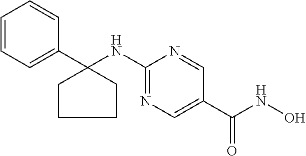 [SMILES: O=C(NO)c1cnc(NC2(c3ccccc3)CCCC2)nc1] | 88 |
| 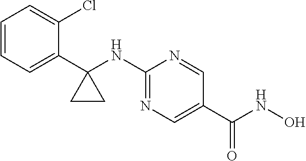 [SMILES: O=C(NO)c1cnc(NC2(c3ccccc3Cl)CC2)nc1] | 90 |
| 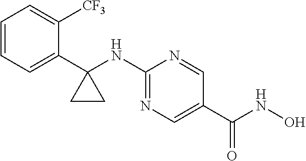 [SMILES: O=C(NO)c1cnc(NC2(c3ccccc3C(F)(F)F)CC2)nc1] | 91 |
| 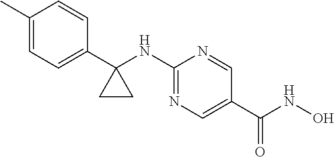 [SMILES: Cc1ccc(C2(Nc3ncc(C(=O)NO)cn3)CC2)cc1] | 93 |
| 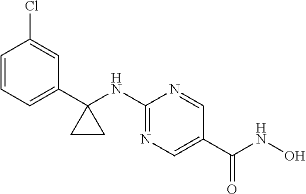 [SMILES: O=C(NO)c1cnc(NC2(c3cccc(Cl)c3)CC2)nc1] | 94 |

and pharmaceutically acceptable salts thereof.

In a further embodiment, the compounds of Formula (I) may be selected from the following:

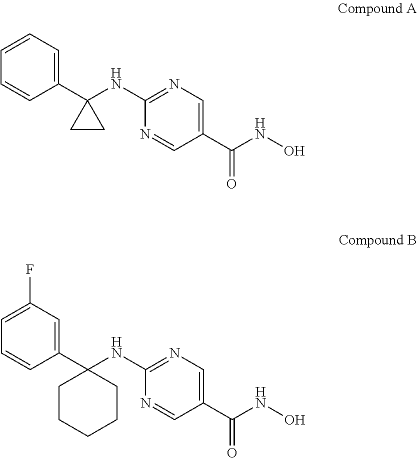 [SMILES: O=C(NO)c1cnc(NC2(c3ccccc3)CC2)nc1 ; O=C(NO)c1cnc(NC2(c3cccc(F)c3)CCCCC2)nc1]

and pharmaceutically acceptable salts thereof.

In an embodiment, the compounds of Formula (I) do not include the following compound:

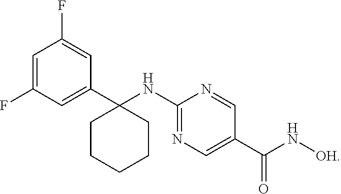 [SMILES: O=C(NO)c1cnc(NC2(c3cc(F)cc(F)c3)CCCCC2)nc1]

The preparation and properties of selective HDAC6 inhibitors according to Formula (I) are provided in International Patent Application No. PCT/US2011/060791 (Publication No. WO/2012/068109), the entire contents of which are incorporated herein by reference.

In an embodiment, the compound of the invention has a selectivity for HDAC6 when tested in a HDAC enzyme assay of about 5 to 1000 fold greater than for other HDACs.

In some embodiments, the compounds described herein are unsolvated. In other embodiments, one or more of the compounds are in solvated form. As known in the art, the solvate can be any of pharmaceutically acceptable solvent, such as water, ethanol, and the like.

Another embodiment is an isotopically labeled compound of any of the compounds delineated herein. Such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

### Methods of the Invention

Histone deacetylases (HDACs) are known to play an essential role in the transcriptional machinery for regulating gene expression, induce histone hyperacetylation and to affect the gene expression. Therefore, HDAC inhibitors are useful as a therapeutic or prophylactic agent for diseases caused by abnormal gene expression such as, for example, inflammatory disorders, diabetes, diabetic complications, homozygous thalassemia, fibrosis, cirrhosis, acute promyelocytic leukemia (APL), organ transplant rejections, autoimmune diseases, protozoal infections, tumors, etc. HDAC inhibition is a promising therapeutic approach for the treatment of a range of central nervous system disorders (Langley B et al., 2005, Current Drug Targets-CNS & Neurological Disorders, 4: 41-50).

In one aspect, provided herein are methods of treating or preventing a peripheral neuropathy in a subject in need thereof comprising administering to the subject a therapeutically effective amount of a compound of Formula (I):

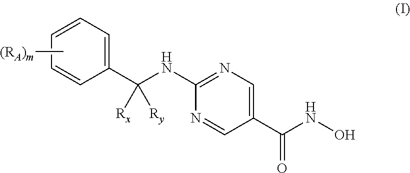

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH or haloalkyl; and
m is 0, 1, or 2.

In an embodiment of the methods provided herein, the compound of Formula (I) is selected from a compound of Table 1 and pharmaceutically acceptable salts thereof. In a further embodiment of the methods provided herein, the compound of Formula (I) is selected from a compound of Table 2 and pharmaceutically acceptable salts thereof.

In a preferred embodiment of the methods provided herein, the compound of Formula (I) is:

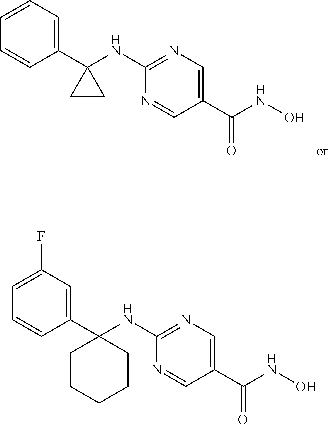 [SMILES: O=C(NO)c1cnc(NC2(c3ccccc3)CC2)nc1 ; "or" ; O=C(NO)c1cnc(NC2(c3cccc(F)c3)CCCCC2)nc1]

or a pharmaceutically acceptable salt thereof.

In one embodiment, the peripheral neuropathy is Charcot-Marie Tooth Disease.

In another embodiment, the peripheral neuropathy is a medication induced peripheral neuropathy.

In a further embodiment, the peripheral neuropathy is chemotherapy-induced peripheral neuropathy. In yet a further embodiment, the chemotherapy-induced peripheral neuropathy is Taxol-induced peripheral neuropathy or vincristine-induced peripheral neuropathy.

The chemotherapy-induced peripheral neuropathy may be associated with various classes of chemotherapeutics including, but not limited to, thalidomide and thalidomide derivatives, epithilones, vinca alkaloids, taxanes, proteasome inhibitors, and platinum-based chemotherapeutics.

Specific chemotherapies associated with peripheral neuropathy include, but are not limited to, cisplatin, carboplatin, oxalaplatin, bortezomib, dicarbazine, thalidomide, lenalidomide, pomalidomide, ixabepilone, streptozocin, syclophosphamide, carmustine, lomustine, procarbazine, mitomyocin, cytarabine, methotrexate, 5-fluorouracil, vinblastine, vincristine, bleomycin, paclitaxel (taxol), asparaginase, busulfan, dacarbazine, fludarabine, hydroxyurea, ifosfamide, mercaptopurine, mitotane, streptozocin, or a mixture of two or more agents thereof.

In another aspect, provided herein are methods of treating or preventing a peripheral neuropathy in a subject in need thereof comprising administering to the subject a therapeutically effective amount of a pharmaceutical composition comprising a compound of Formula (I), to thereby treat or prevent the peripheral neuropathy.

In one aspect, provided herein are methods of treating or preventing a peripheral neuropathy in a subject in need thereof comprising administering to the subject a therapeutically effective amount of a compound of Table 1 or a pharmaceutical composition comprising a compound of Table 1, to thereby treat or prevent the peripheral neuropathy.

In one embodiment, the peripheral neuropathy is Charcot-Marie Tooth Disease.

In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy.

In one aspect, provided herein are methods of treating or preventing a peripheral neuropathy in a subject in need thereof comprising administering to the subject a therapeutically effective amount of a compound of Table 2 or a pharmaceutical composition comprising a compound of Table 2, to thereby treat or prevent the peripheral neuropathy.

In one embodiment, the peripheral neuropathy is Charcot-Marie Tooth Disease.

In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy.

In one aspect, provided herein are methods of treating or preventing a peripheral neuropathy in a subject in need thereof comprising administering to the subject a therapeutically effective amount of the compound:

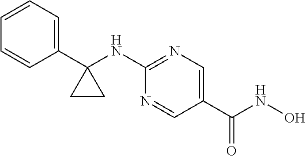 [SMILES: O=C(NO)c1cnc(NC2(c3ccccc3)CC2)nc1]

or a pharmaceutical composition comprising the compound, to thereby treat or prevent the peripheral neuropathy.

In one embodiment, the peripheral neuropathy is Charcot-Marie Tooth Disease.

In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy.

In one aspect, provided herein are methods of treating or preventing a peripheral neuropathy in a subject in need thereof comprising administering to the subject a therapeutically effective amount of the compound:

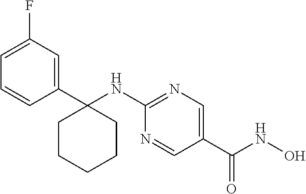 [SMILES: O=C(NO)c1cnc(NC2(c3cccc(F)c3)CCCCC2)nc1]

or a pharmaceutical composition comprising the compound, to thereby treat or prevent the peripheral neuropathy.

In one embodiment, the peripheral neuropathy is Charcot-Marie Tooth Disease.

In another embodiment, the peripheral neuropathy is chemotherapy-induced peripheral neuropathy. In yet a further embodiment, the chemotherapy-induced peripheral neuropathy is taxol-induced peripheral neuropathy or vincristine-induced peripheral neuropathy.

In certain embodiments, the invention provides a method of treatment of any of the disorders described herein, wherein the subject is a human.

In accordance with the foregoing, the present invention further provides a method for preventing or treating any of the diseases or disorders described above in a subject in need of such treatment, which method comprises administering to said subject a therapeutically effective amount of a compound of the invention or a pharmaceutically acceptable salt thereof. For any of the above uses, the required dosage will vary depending on the mode of administration, the particular condition to be treated and the effect desired.

According to the methods of treatment of the present invention, disorders are treated or prevented in a subject, such as a human or other animal, by administering to the subject a therapeutically effective amount of a compound of the invention, in such amounts and for such time as is necessary to achieve the desired result. The term “therapeutically effective amount” of a compound of the invention means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

In certain embodiments, a therapeutic amount or dose of the compounds of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Pharmaceutical Compositions

In another aspect, the invention provides a pharmaceutical composition comprising any of the compounds of the instant invention (compounds of Formula (I) or Compound A or B) or pharmaceutically acceptable salts thereof, together with a pharmaceutically acceptable carrier.

The pharmaceutical compositions of the present invention comprise a therapeutically effective amount of a compound of the present invention formulated together with one or more pharmaceutically acceptable carriers. The term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Compounds of the invention can be administered as pharmaceutical compositions by any conventional route, in particular enterally, for example, orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

### EXAMPLES

Examples have been set forth below for the purpose of illustration and to describe certain specific embodiments of the invention. However, the scope of the claims is not to be in any way limited by the examples set forth herein. Various changes and modifications to the disclosed embodiments will be apparent to those skilled in the art and such changes and modifications including, without limitation, those relating to the chemical structures, substitutents, derivatives, formulations and/or methods of the invention may be made without departing from the spirit of the invention and the scope of the appended claims.

### Example 1

### Synthesis of N-hydroxy-2-((1-phenylcyclopropyl)amino)pyrimidine-5-carboxamide (Compound A)

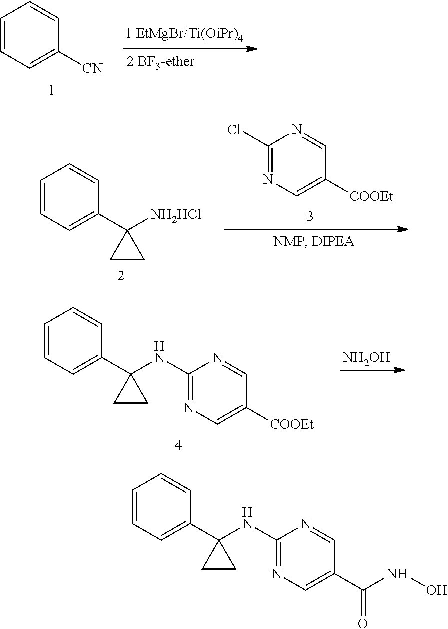

Synthesis of Intermediate 2:

A solution of compound 1, benzonitrile, (250 g, 1.0 equiv.), and Ti(OiPr)₄ (1330 ml, 1.5 equiv.) in MBTE (3750 ml) was cooled to about −10 to −5° C. under a nitrogen atmosphere. EtMgBr (1610 ml, 3.0M, 2.3 equiv.) was added dropwise over a period of 60 min., during which the inner temperature of the reaction was kept below 5° C. The reaction mixture was allowed to warm to 15-20° C. for 1 hr. BF₃-ether (1300 ml, 2.0 equiv.) was added dropwise over a period of 60 min., while the inner temperature was maintained below 15° C. The reaction mixture was stirred at 15-20° C. for 1-2 hr. and stopped when a low level of benzonitrile remained. 1N HCl (2500 ml) was added dropwise while maintaining the inner temperature below 30° C. NaOH (20%, 3000 ml) was added dropwise to bring the pH to about 9.0, while still maintaining a temperature below 30° C. The reaction mixture was extracted with MTBE (3 L×2) and EtOAc (3 L×2), and the combined organic layers were dried with anhydrous Na₂SO₄ and concentrated under reduced pressure (below 45° C.) to yield a red oil. MTBE (2500 ml) was added to the oil to give a clear solution, and upon bubbling with dry HCl gas, a solid precipitated. This solid was filtered and dried in vacuum yielding 143 g of compound 2.

Synthesis of Intermediate 4:

Compound 2 (620 g, 1.0 equiv) and DIPEA (1080 g, 2.2 equiv. were dissolved in NMP (3100 ml) and stirred for 20 min. Compound 3 (680 g, 1.02 equiv.) was added and the reaction mixture was heated to about 85-95° C. for 4 hrs. The solution was allowed to slowly cool to r.t. This solution was poured onto H₂O (20 L) and much of the solid was precipitated out from the solution with strong stirring. The mixture was filtered and the cake was dried under reduced pressure at 50° C. for 24 hr., yielding 896 g of compound 4 (solid, 86.8%).

### Synthesis of N-hydroxy-2-((1-phenylcyclopropyl)amino)pyrimidine-5-carboxamide (Compound A)

A solution of MeOH (1000 ml) was cooled to about 0-5° C. with stirring. NH₂OH HCl (1107 g, 10 equiv.) was added, followed by careful addition of NaOCH₃ (1000 g, 12.0 equiv.) The resulting mixture was stirred at 0-5° C. for one hr, and was filtered to remove the solid. Compound 4 (450 g, 1.0 equiv.) was added to the reaction mixture in one portion, and stirred at 10° C. for two hours until compound 4 was consumed. The reaction mixture was adjusted to a pH of about 8.5-9 through addition of HCl (6N), resulting in precipitation. The mixture was concentrated under reduced pressure. Water (3000 ml) was added to the residue with intense stirring and the precipitate was collected by filtration. The product was dried in an oven at 45° C. overnight (340 g, 79% yield).

### Example 2

### Synthesis of 2-((1-(3-fluorophenyl)cyclohexyl)amino)-N-hydroxypyrimidine-5-carboxamide (Compound B)

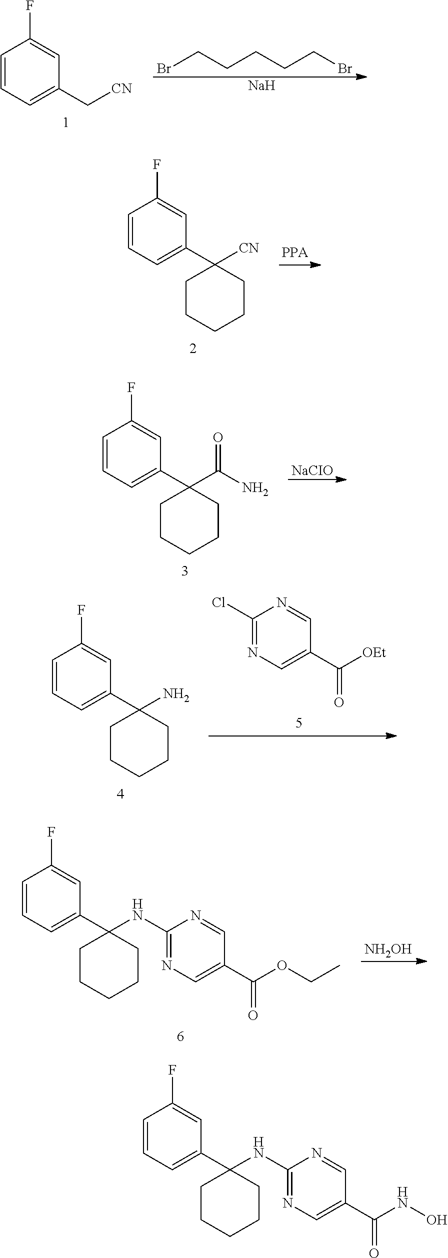

Synthesis of Intermediate 2:

To a solution of compound 1 (100 g, 0.74 mol) in dry DMF (1000 ml) was added 1,5-dibromopentane (170 g, 0.74 mol). NaH (65 g, 2.2 eq) was added dropwise while the reaction was cooled in an ice bath. The resulting mixture was vigorously stirred overnight at 50° C. The suspension was carefully quenched with ice water and extracted with ethyl acetate (3×500 ml). The combined organic layers were concentrated to afford the crude product, which was purified by flash column chromatography to give compound 2 as pale solid (100 g, 67%).

Synthesis of Intermediate 3:

A solution of compound 2 (100 g, 0.49 mol) in PPA (500 ml) was heated at 110° C. for about 5-6 hours. After completion, the resulting mixture was carefully adjusted to a pH of about 8-9 with sat.NaHCO₃ solution. The resulting precipitate was collected and washed with water (1000 ml) to afford compound 3 as white solid (95 g, 87%).

Synthesis of Intermediate 4:

To a solution of compound 3 (95 g, 0.43 mol) in n-BuOH (800 ml) was added NaClO (260 ml, 1.4 eq). 3N NaOH (400 ml, 2.8 equiv.) was then added at 0° C. and the reaction was stirred overnight at r.t. The resulting mixture was extracted with EA (2×500 ml), and the combined organic layers washed with brine. The solvent was removed in vacuo to afford the crude product which was further purified by treatment with HCl salt to yield compound 4 as a white powder (72 g, 73%).

Synthesis of Intermediate 6:

To a solution of compound 4 (2.29 g 10 mmol) in dioxane (50 ml) was added compound 5 (1.87 g, 1.0 equiv.) and DIPEA (2.58 g, 2.0 equiv.). The mixture was heated overnight at 110-120° C. The resulting mixture was directly purified on silica gel column to afford the coupled product, compound 6, as a white solid (1.37 g, 40%).

### Synthesis of 2-((1-(3-fluorophenyl)cyclohexyl)amino)-N-hydroxypyrimidine-5-carboxamide (Compound B)

To a solution of compound 6 (100 mg, 0.29 mmol) in MeOH/DCM (10 ml, 1:1) was added 50% NH₂OH in water (2 ml, excess). Sat. NaOH in MeOH (2 ml, excess) was then added at 0° C. and the reaction was stirred for 3-4 hours. After completion, the resulting mixture was concentrated and acidified with 2N HCl to reach a pH of 4-5. The precipitate was collected and washed with water (10 ml) to remove excess NH₂OH. Drying the precipitate afforded 2-((1-(3-fluorophenyl)cyclohexyl)amino)-N-hydroxypyrimidine-5-carboxamide as a white powder (70 mg, 73%).

### Example 3

### HDAC Enzyme Assays

Compounds for testing were diluted in DMSO to 50 fold the final concentration and a ten point three fold dilution series was made. The compounds were diluted in assay buffer (50 mM HEPES, pH 7.4, 100 mM KCl, 0.001% Tween-20, 0.05% BSA, 201 μM TCEP) to 6 fold their final concentration. The HDAC enzymes (purchased from BPS Biosciences) were diluted to 1.5 fold their final concentration in assay buffer. The tripeptide substrate and trypsin at 0.051 μM final concentration were diluted in assay buffer at 6 fold their final concentration. The final enzyme concentrations used in these assays were 3.3 ng/ml (HDAC1), 0.2 ng/ml (HDAC2), 0.08 ng/ml (HDAC3) and 2 ng/ml (HDAC6). The final substrate concentrations used were 16 μM (HDAC1), 101 μM (HDAC2), 171 μM (HDAC3) and 141 μM (HDAC6). Five μl of compound and 20 μl of enzyme were added to wells of a black, opaque 384 well plate in duplicate. Enzyme and compound were incubated together at room temperature for 10 minutes. Five μl of substrate was added to each well, the plate was shaken for 60 seconds and placed into a Victor 2 microtiter plate reader. The development of fluorescence was monitored for 60 min and the linear rate of the reaction was calculated. The IC₅₀ was determined using Graph Pad Prism by a four parameter curve fit. The IC₅₀ values for Compounds A and B are shown below in Table 3.

### Example 4

### Rat Pharmacokinetic Studies

Male SD rats were fasted overnight. Compounds A and B were dissolved in dimethyl acetamide at 10 times the final concentration, then Solutol HS 15 (BASF) was added to a final concentration of 10%. Finally 80% saline was added and vortexed to achieve a clear solution. For the IV dosing three animals were injected via the foot dorsal vein with 1 mg/kg compound. For the PO dosing 5 mg/kg of compound was delivered by oral gavage. Blood was collected via the tail vein into K2EDTA tubes at 5 minutes, 15 minutes, 30 minutes, 1 hour, 2 hours, 4 hours, 8 hours and 24 hours after dosing. The blood was centrifuged at 2000 g for 5 minutes at 4° C. to obtain plasma. The plasma was extracted with acetonitrile and the level of compound was analyzed by LC/MS/MS. The level of compound in plasma was calculated from a standard curve in rat plasma. The IV clearance and area under the curve were calculated using WinNonLin software. The dose adjusted area under the curve for the IV and oral dosing were used to calculate the oral bioavailability. A summary of results is presented in Table 3.

### Example 5

### Mouse Pharmacokinetic Studies

Male C57BL/6 mice were fasted overnight. Compounds A and B were dissolved in dimethyl acetamide at 10 times the final concentration, then Solutol HS 15 (BASF) was added to a final concentration of 10%. Finally 80% saline was added and vortexed to achieve a clear solution. Fifteen animals were injected via the tail vein with 1 mg/kg compound. Blood was collected by retro-orbital bleed at 5 minutes, 15 minutes, 30 minutes, 1 hour, 2 hours, 4 hours, 8 hours and 24 hours after dosing. At 5 minutes, 30 minutes, 1 hour and 4 hours after dosing three animals per time point were sacrificed and brains were removed. The blood was centrifuged at 2000 g for 5 minutes at 4° C. to obtain plasma. Brain samples were homogenized in PBS. The plasma and brain homogenate were extracted with acetonitrile and the level of compound was analyzed by LC/MS/MS. The level of compound in plasma was calculated from a standard curve in rat plasma and the level in brain was calculated from a standard curve in brain homogenate. The area under the curve in plasma and brain were calculated using WinNonLin software. The brain to plasma ratio was determined using the area under the curve values for the two compartments. A summary of the results is presented in Table 3.

TABLE 3

| | HDAC1 | HDAC2 | HDAC3 | HDAC6 | Rat IV | Rat Oral | Mouse |
|---|---|---|---|---|---|---|---|
| | IC50 | IC50 | IC50 | IC50 | Clearance | Bioavailability | plasma/brain |
| Compound | (nM) | (nM) | (nM) | (nM) | (L/hr/kg) | (%) | ratio |
| Compound A | 94 | 128 | 158 | 1.7 | 6.0 | 16.5 | 1.23 |
| Compound B | 2123 | 2570 | 11223 | 7.5 | 2.3 | 7.9 | 1.28 |

Inhibition of HDAC1, 2 and 3 have been associated with toxicity, such as thrombocytopenia, neutropenia, anemia, and fatigue. A compound with a higher selectivity for HDAC6 over HDACs 1, 2, and 3 would therefore be expected to have a larger therapeutic window.

For use in therapy, compounds must have a low IV clearance rate (to maintain biologically active concentrations in plasma and tissues for a prolonged time), a high oral bioavailability (to allow consistent delivery of compound by the oral route) and a high blood brain barrier penetration (to allow compound to reach the relevant target in the brain).

### Example 6

### Compound A in Vincristine-Induced Neuropathy Study

A study was conducted to assess the analgesic activity of Compound A at doses of 3 mg/kg (group 3) and 10 mg/kg (group 4) in the vincristine-induced neuropathic pain model in rats.

Methods:

Vincristine Treatment:

Neuropathic pain is induced by injecting 5 ml/kg Vincristine (50 μg/kg) administered daily IP for a period of 12 days. The development of neuropathic pain is confirmed by measuring of mechanical allodynia (Von Frey test) on study day 13.

Pain Response Evaluation (Von Frey Testing):

Allodynic response to tactile stimulation is assessed using the Von Frey apparatus (Touch Test®).

The rat will be placed in an enclosure and positioned on a metal mesh surface, but allowed to move freely. The rats' cabins are covered with red cellophane to diminish environmental disturbances. The test begins after a cessation of exploratory behavior. The set of Von Frey monofilaments provide an approximate logarithmic scale of actual force and a linear scale of perceived intensity.

The operating principle: when the tip of a fiber of given length and diameter is pressed against the skin at right angles, the force of application increases as long as the researcher continues to advance the probe until the fiber bends. After the fiber bends, the probe continues to advance, causing the fiber to bend more, but without additional force being applied. Rodents exhibit a paw withdrawal reflex when the paw is unexpectedly touched. The Touch Test™ Sensory Evaluator can be used on the plantar surfaces of the rat's foot. The animal indicates sensation by pulling back its paw. The minimal force needed to elevate the withdrawal reflex is considered/designated as the value of reference. In order to achieve paw withdrawal, the pressure applied is sometimes greater than 60 g, often requiring the researcher to apply enough pressure with the Von Frey filament to actually lift the paw of the naive animal. Decreases in force needed to induce withdrawal are indicative of allodynia, as the force applied is a non-painful stimulus under normal conditions.

### Dose Groups

| Group | Group | | | | | Administration |
|---|---|---|---|---|---|---|
| No. | Size | Test Item | Dose | Volume | Route | Regime |
| 1 | N = 10 | Vehicle | N/A | N/A | P0 | Twice a day (b.i.d) |
| | | | | | | from |
| | | | | | | study day 7 through |
| | | | | | | day 12. |
| | | | | | | On days 12-15 twice a |
| | | | | | | day (b.i.d) 2 hours |
| | | | | | | prior to Von Frey |
| | | | | | | testing. |
| 2 | N = 10 | Gabapentin | 150 mg/kg | 5 ml/kg | IP | Once, 2 hours prior to |
| | | | | | | testing on study days |
| | | | | | | 12, 13, 14 and 15. |
| 3 | N = 10 | Compound A | 3 mg/kg | 5 ml/kg | P0 | Twice a day (b.i.d) |
| 4 | N = 10 | Compound A | 10 mg/kg | 5 ml/kg | P0 | from |
| | | | | | | study day 7 through |
| | | | | | | day 12. |
| | | | | | | On days 12-15 twice a |
| | | | | | | day (b.i.d) 2 hours |
| | | | | | | prior to Von Frey |
| | | | | | | testing. |

Summary of Results:

Animals were dosed b.i.d with either Vehicle or Test Item starting from day 7 through the end of the study (day 15). Gabapentin (Group 2), the positive control, dosed once on study days 13-15 prior to Von Frey testing, was active in reversing pain significantly vs. vehicle treated group (Group 1) on all tested days: 43.95±5.00 g vs. 14.50±1.51 g for the vehicle group on day 15; p<0.05.

Treatment with Compound A at a dose of 3 mg/kg (group 3) was not active in increasing the withdrawal force. Treatment with Compound A at a dose of 10 mg/kg (group 4) was not effective following a single treatment (study day 13, 11.00±1.07 g ns. vs. 9.45±0.66 g for the vehicle). On the next day following 3 doses (two doses (AM and PM) on study day 13 and AM dose on study day 14) a significant effect was recorded in reversing pain: 33.85±6.96 g vs. 14.50±1.51 g for the Vehicle (Group 1) on study day 15; p<0.05.

*p<0.05 vs. Vehicle using one-way ANOVA followed by Tukey test.

In view of the findings obtained under the conditions of this study, and confined to the in-life data, treatments with Compound A at doses of 3 mg/kg and 10 mg/kg PO starting from study day 7 showed trend of improvement of animals waking ability. Treatment with Compound A at the dose of 10 mg/kg also significantly decreased the Von Frey force on study days 14-15.

### Example 7

### Compound A in Taxol-Induced Neuropathy

A study was conducted to extend the previous findings in the vincristine model to a different model of chemotherapy-induced neuropathy, the taxol-induced neuropathic pain model in rats.

Methods:

Taxol Treatment:

Neuropathic pain is induced by a 0.5 ml/kg injection of taxol (2 mg/kg) administered IP on days 0-12. The development of neuropathic pain is confirmed by measuring mechanical allodynia (Von Frey test) on study day 13 as described above.

### Dose Groups:

| Group | Group | | | | | Administration |
|---|---|---|---|---|---|---|
| No. | Size | Test Item | Dose | Volume | Route | Regime |
| 1 | N = 12 | Vehicle | N/A | N/A | P0 | Twice a day (b.i.d) |
| | | | | | | from |
| | | | | | | study day 7 through |
| | | | | | | day 12. |
| | | | | | | On days 12-15 twice |
| | | | | | | a day (b.i.d) 2 hours |
| | | | | | | prior to Von Frey |
| | | | | | | testing. |
| 2 | N = 10 | Gabapentin | 150 mg/kg | 5 ml/kg | IP | Once, 2 hours prior to |
| | | | | | | testing on study days |
| | | | | | | 12, 13, 14 and 15. |
| 3 | N = 10 | Compound A | 3 mg/kg | 5 ml/kg | P0 | Twice a day (b.i.d) |
| 4 | N = 9 | Compound A | 10 mg/kg | 5 ml/kg | P0 | from |
| | | | | | | study day 7 through |
| | | | | | | day 12. |
| | | | | | | On days 12-15 twice |
| | | | | | | a day (b.i.d) 2 hours |
| | | | | | | prior to Von Frey |
| | | | | | | testing. |

Summary of Results:

Pain was assessed using Von-Frey (VF) test for tactile allodynia on study days 13-15. The Rotarod Test was used to assess motor/coordination function.

Neuropathic pain was induced by a 0.5 ml/kg injection of taxol (2 mg/kg) administered IP on study days 0-12. The development of neuropathic pain was confirmed by measuring mechanical allodynia (Von Frey test) on study day 13.

Following injection with taxol animals experienced pain as was assessed using the Von Frey test. The reduction in withdrawal force was noticed on study day 13. This low withdrawal force was also observed for the vehicle treated group on the last day of the study (study day 15): 13.21±1.49 g vs. 60.00±0.00 g at baseline (p<0.05).

Gabapentin at a dose of 150 mg/kg (Group 2) significantly reversed neuropathic pain vs. vehicle 2 hours post dosing on study days 13-15: 54.90±3.63 g vs. 13.21±1.49 g for the vehicle group on study day 15 (p<0.05).

Compound A at a dose of 3 mg/kg (Group 3) significantly reversed neuropathic pain vs. vehicle 2 hours post dosing on study days 13-15: 53.20±2.78 g vs. 13.21±1.49 g for the vehicle group on study day 15 (p<0.05).

Compound A at a dose of 10 mg/kg (Group 4) completely reversed neuropathic pain vs. vehicle 2 hours prior and post dosing on all tested days: 53.11±4.77 g vs. 15.33±2.13 g for the vehicle group on study day 13 post dosing (p<0.05).

In view of the findings obtained under the conditions of this study, and confined to the in-life data, it was concluded that Compound A at doses of 3 mg/kg and 10 mg/kg was active in reversing pain.

### Example 8

### Further Studies Showing Compounds A and B are Potent and Selective HDAC6 Inhibitors

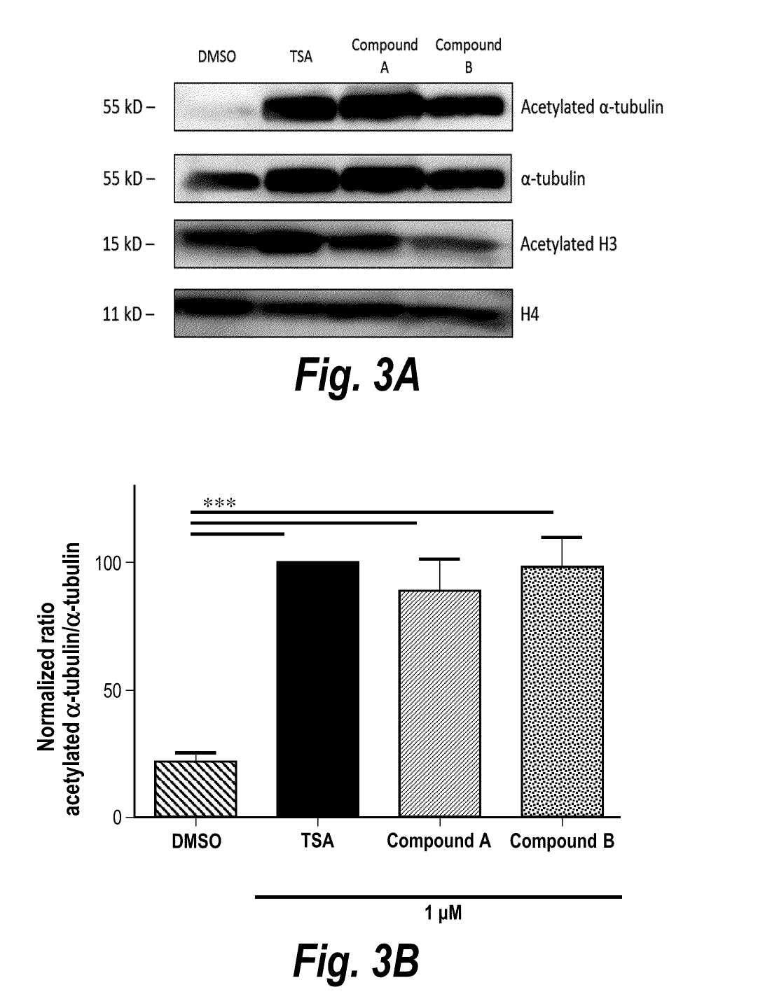

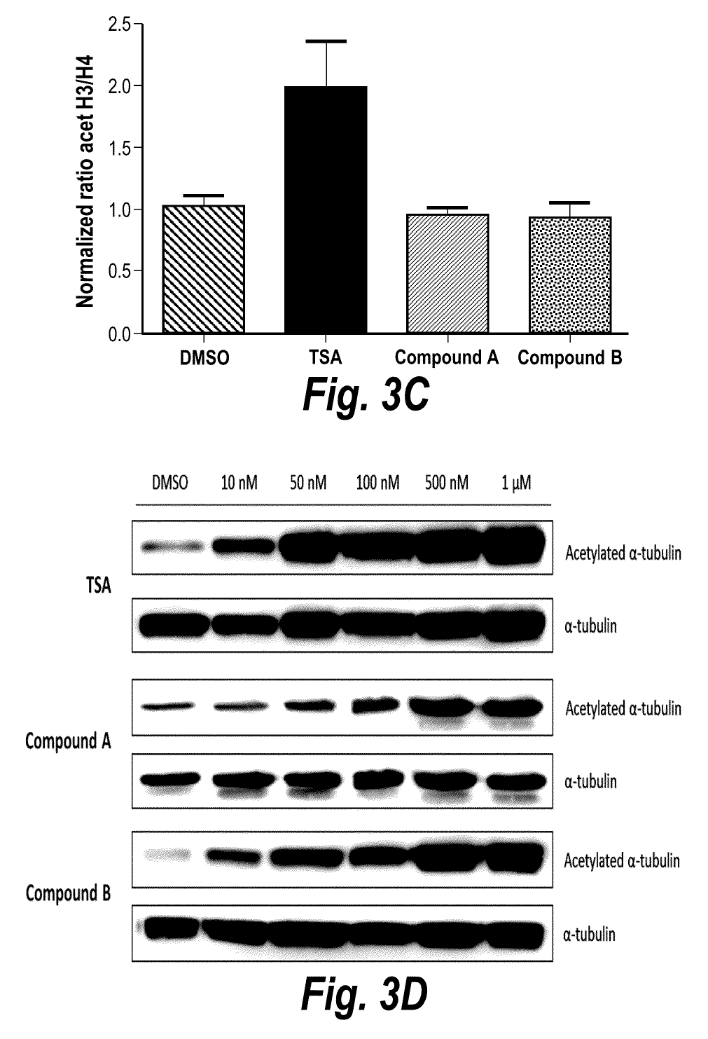

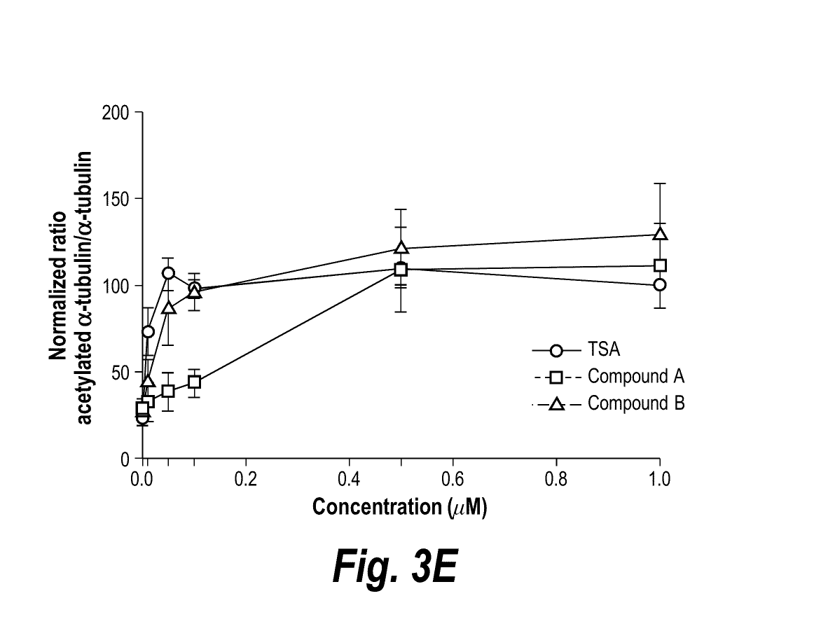

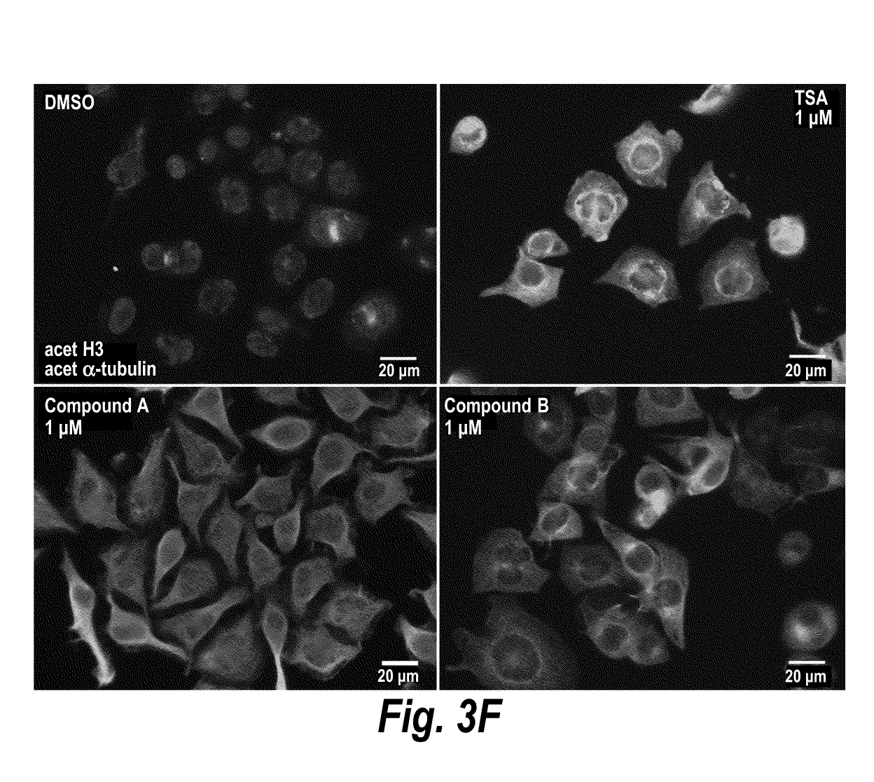

Compounds A and B were also tested in a mouse neuroblastoma cell line, Neuro2a, for their potency and selectivity to inhibit HDAC6. The potency was evaluated by checking the levels of acetylated α-tubulin on Western Blot (WB) while the selectivity of the compounds was determined by the acetylation status of histone 3 (H3). Compared to DMSO, 1 μM of both Compound A and Compound B were able to induce a significant increase of the acetylated α-tubulin levels similar to the effect of Trichostatin A (TSA), which was used as a positive control (FIGS. 3A and 3B). However, neither compounds affected the levels of acetylated H3 indicating that they are not inhibiting other members of the HDAC family (FIG. 3C). Staining of treated Neuro2a cells confirmed the increased levels of acetylated α-tubulin induced by TSA, Compound A, and Compound B, while more acetylated H3 was found only in the nuclei of TSA-treated cells (FIG. 3F). In a next step, different concentrations of the compounds were tested to gain more insights into the kinetics. WB analysis shows that both TSA and Compound B work at lower concentrations and therefore are more potent than the Compound A (FIGS. 3D and 3E). However, at 500 nM all the compounds are able to increase the acetylated tubulin levels to a similar extent (FIG. 3E).

### Example 9

### Mitochondrial Transport Defects can be Rescued by Compounds A and B in a Charcot-Marie Tooth Model

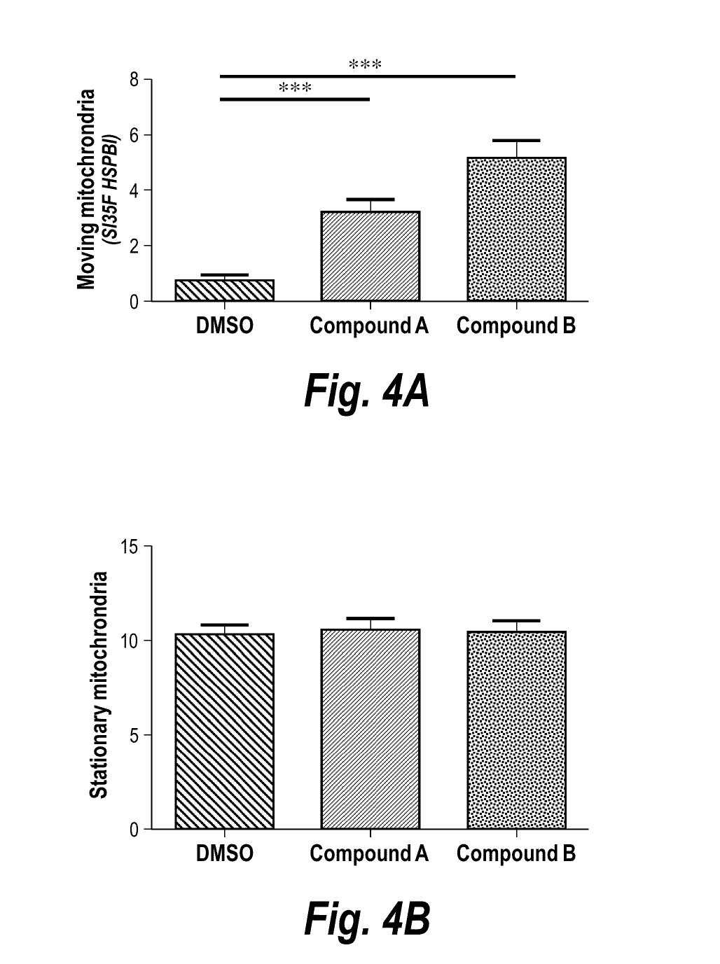

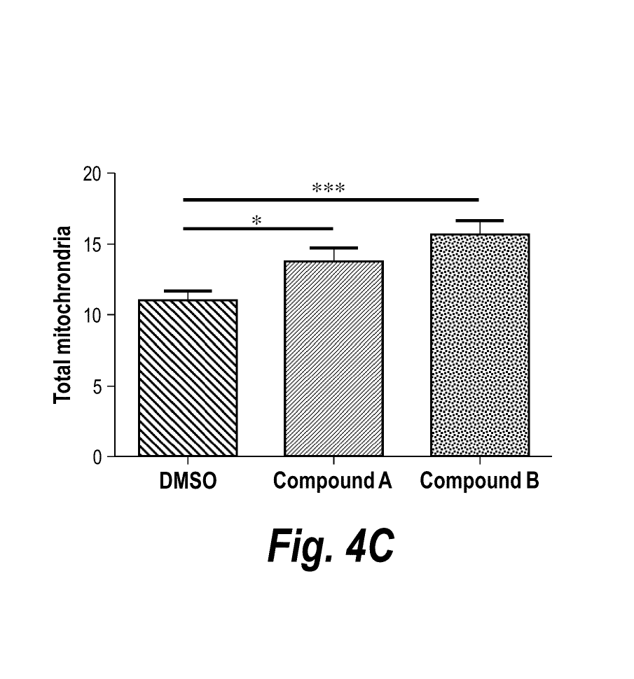

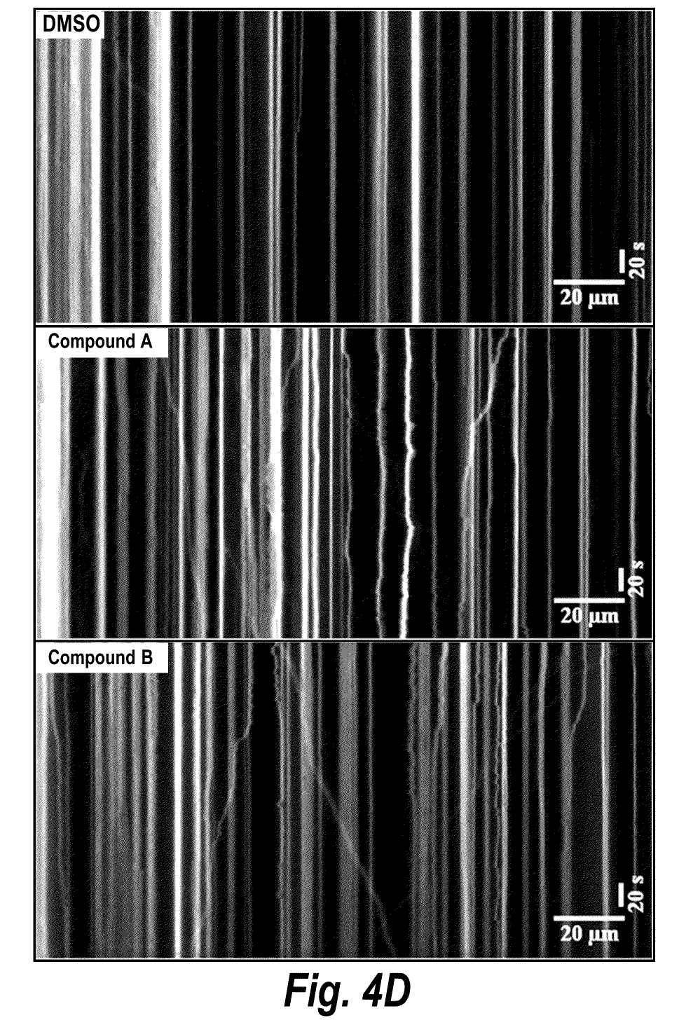

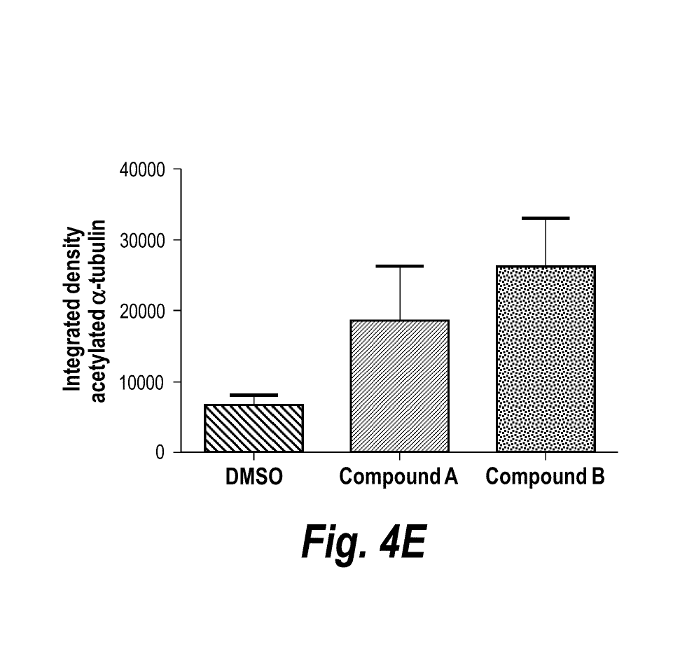

As both compounds have been shown to be potent and selective inhibitors of HDAC6, it was addressed whether they were able to restore the defects seen in the mutant HSPB1-induced CMT2 mouse model (d'Ydewalle et al., 2011, HDAC6 inhibitors reverse axonal loss in a mouse model recapitulating mutant HSPB1-induced Charcot-Marie-Tooth disease. Nature medicine. 17:968-974). The axonal transport of mitochondria in dorsal root ganglion (DRG) neurons cultured from symptomatic HSPB1^{S135F} mice has been shown to be disturbed (FIG. 4D, upper kymograph). This defect could be rescued by the selective inhibition of HDAC6 by Tubastatin A (d'Ydewalle et al., 2011). Therefore, DRG neurons were cultured from symptomatic (12 month old) HSPB1^{S135F} mice and treated with the highest dose tested in NTG DRG neurons (2.5 μM) to test the ability of Compound A and Compound B to restore the axonal transport defects. Both compounds were able to increase the number of moving mitochondria in the neurites of HSPB1^{S135F} DRG neurons (FIGS. 4A and 4D). The number of total mitochondria also increased compared to DMSO treated cells (FIG. 4C) while the amount of stationary mitochondria remained unchanged (FIG. 4B). Staining of the treated DRG neurons showed that this rescue correlated with an increase in the acetylation of α-tubulin in the neurites (FIG. 4E).

### Example 10

### Effect of Compounds A and B on Motor and Sensory Deficits of Mutant HSPB1^{S135F} Mice

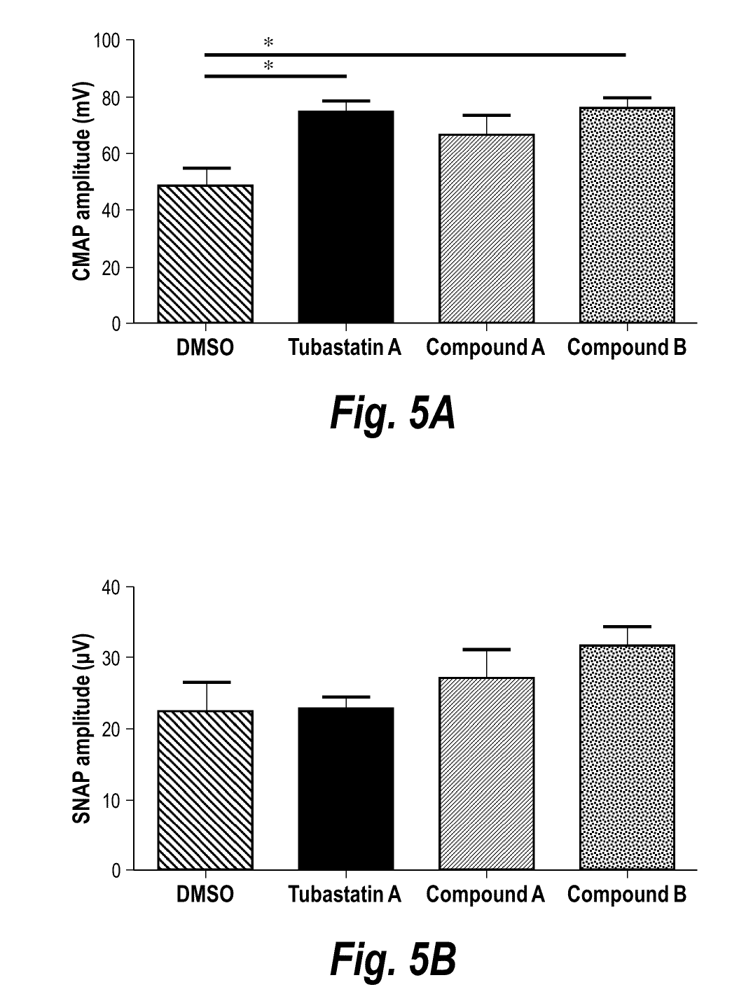

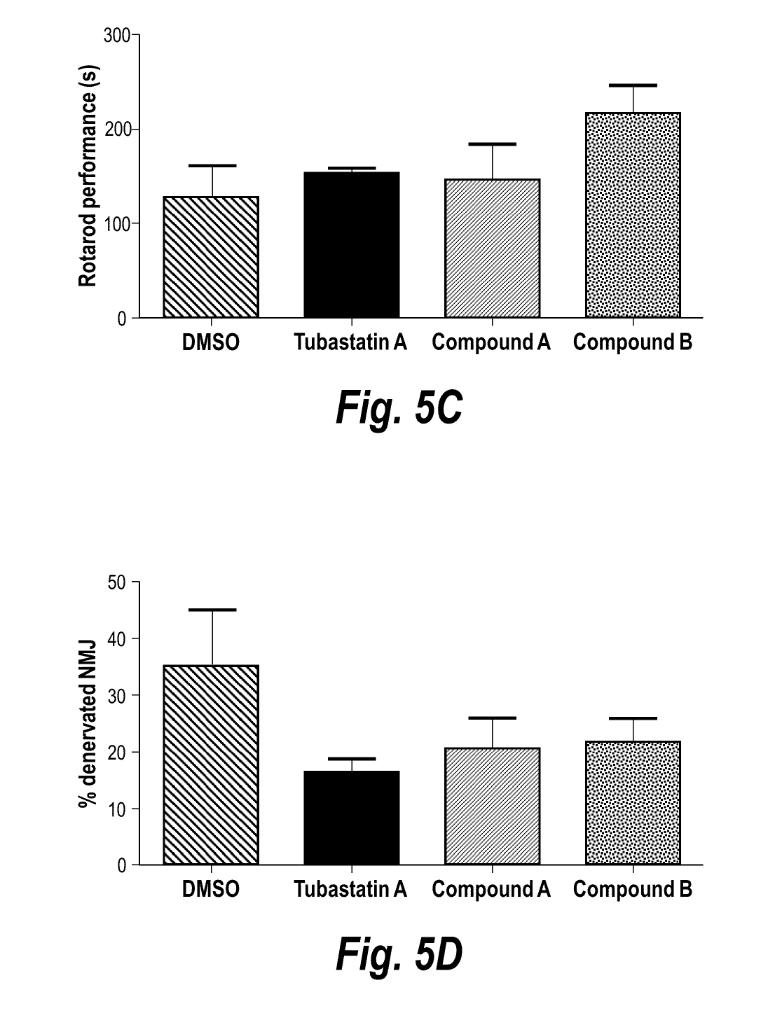

From the age of 6 months on, mutant HSPB1^{S135F} mice display motor and sensory deficits (d'Ydewalle et al., 2011). These mice display a decreased motor performance when placed on an accelerating rotarod and the amplitudes of the motor and sensory nerves are severely reduced (FIGS. 5A-C). Moreover, the innervation of neuromuscular junctions in the gastrocnemius muscle is also reduced (FIG. 5D). These defects can be restored by HDAC6 inhibition by using trichostatin A (TSA) or Tubastatin A (d'Ydewalle et al., 2011). In order to compare the effect of Compound A and Compound B with the effect of Tubastatin A, symptomatic animals were treated with either a low concentration (3 mg/kg) of Tubastatin A, Compound A or Compound B and checked for their ability to restore the motor and sensory defects. A statistical significant rescue of the CMAP amplitude was observed by treating the mutant HSPB1^{S135F} mice with Compound B, both for the rescue of the SNAP amplitude as well as for the reinnervation of neuromuscular junctions. A positive trend for all the compounds tested was observed.

### Example 11

### Compounds A and B Rescue Vincristine-Induced Sensory Peripheral Neuropathies In Vivo

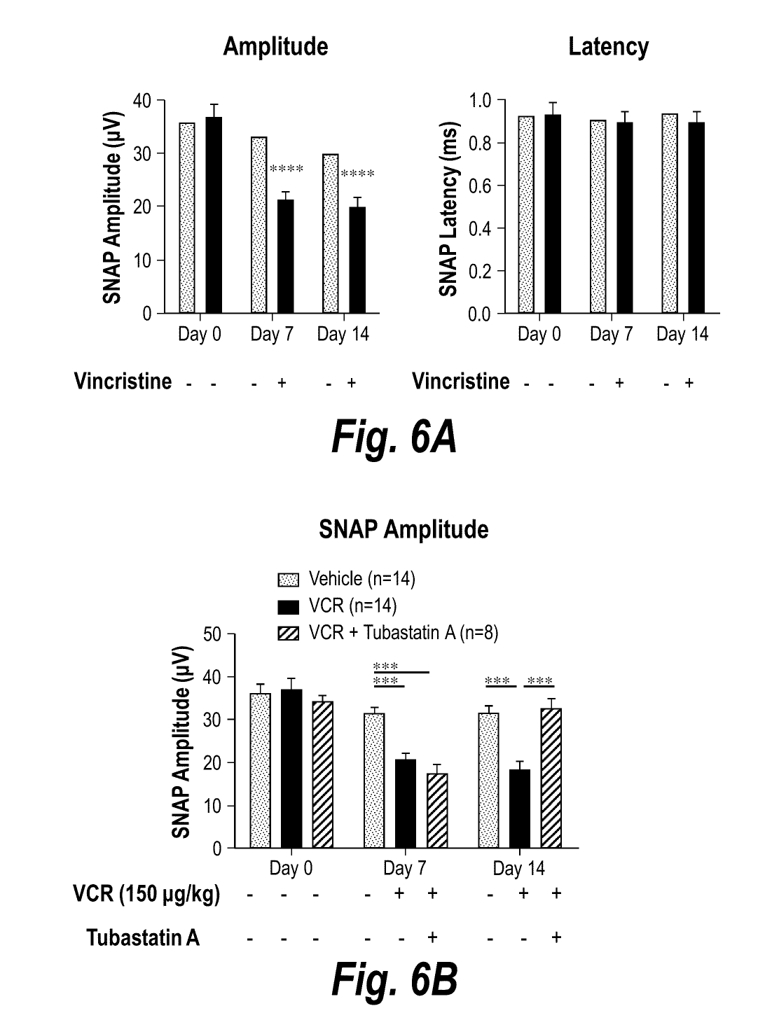

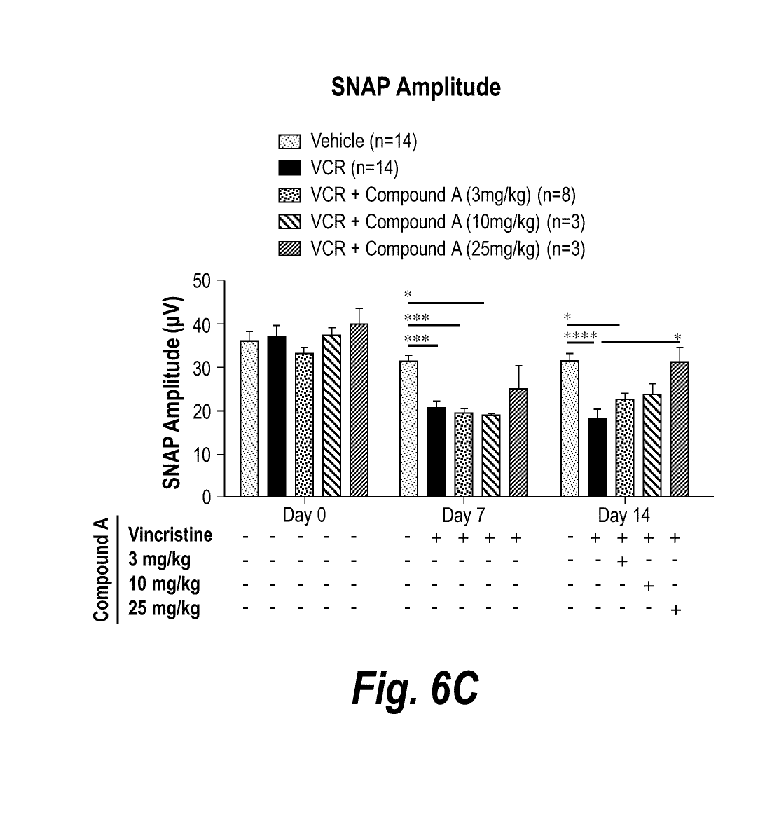

To study whether treatment with vincristine in vivo could induce a peripheral neuropathy, C57B16/J mice were injected intraperitoneally with vincristine on a daily basis. First, the animals were injected with different doses of vincristine. As sensory rather than motor problems are more pronounced in vincristine associated neuropathy patients, the Sensory Nerve Action Potentials (SNAPs) of the dorsal caudal tail nerve were first evaluated (FIG. 6A). After 7 consecutive injections, vincristine-induced a reduction in SNAP amplitude of 35.4% compared to vehicle treated mice, and this reduction did not further increase over time. In contrast, the SNAP latencies were unaffected by vincristine treatment (FIG. 6A), which suggests the peripheral neuropathy is axonal of nature, while myelination is not altered.

These animals were co-treated with vincristine and a selective HDAC6-inhibitor (Tubastatin A) to study the therapeutic potential of HDAC6-inhibition. Daily intraperitoneal injection of Tubastatin A (25 mg/kg) restored the reduction in SNAP amplitudes induced by vincristine completely after 1 week of treatment (FIG. 6B). Next, the effect of Compound A at three different doses on the sensory neuropathy induced by vincristine was investigated (FIG. 6C). Similarly to Tubastatin A, Compound A seems to reduce the sensory neuropathy after one week of intraperitoneal treatment at a dosage of 25 mg/kg/day (FIG. 6C). A motor phenotype in our mice after vincristine treatment was not detected (results not shown). These data imply vincristine induces an axonal neuropathy in the sensory nerves and specific inhibition of HDAC6 can restore this phenotype.

### INCORPORATION BY REFERENCE

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A method of treating a peripheral neuropathy in a subject in need thereof comprising administering to the subject a therapeutically effective amount of a compound of Formula (I): 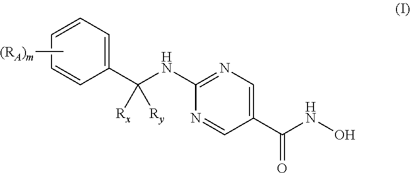
or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH or haloalkyl; and
m is 0, 1, or 2.
or a pharmaceutically acceptable salt thereof, to thereby treat or prevent the peripheral neuropathy.

2. The method of claim 1, wherein the peripheral neuropathy is Charcot-Marie Tooth Disease.

3. The method of claim 1, wherein the peripheral neuropathy is chemotherapy-induced peripheral neuropathy.

4. The method of claim 3, wherein the chemotherapy-induced peripheral neuropathy is taxol-induced peripheral neuropathy or vincristine-induced peripheral neuropathy.

5. A method of treating a peripheral neuropathy in a subject in need thereof comprising administering to the subject a therapeutically effective amount of the compound: 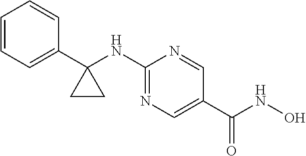 [SMILES: O=C(NO)c1cnc(NC2(c3ccccc3)CC2)nc1]
or a pharmaceutical acceptable salt thereof, to thereby treat or prevent the peripheral neuropathy.

6. The method of claim 5, wherein the peripheral neuropathy is Charcot-Marie Tooth Disease.

7. The method of claim 5, wherein the peripheral neuropathy is chemotherapy-induced peripheral neuropathy.

8. The method of claim 7, wherein the chemotherapy-induced peripheral neuropathy is taxol-induced peripheral neuropathy or vincristine-induced peripheral neuropathy.

9. A method of treating a peripheral neuropathy in a subject in need thereof comprising administering to the subject a therapeutically effective amount of the compound: 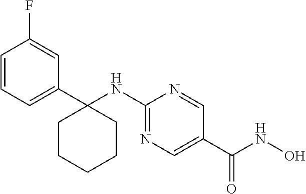 [SMILES: O=C(NO)c1cnc(NC2(c3cccc(F)c3)CCCCC2)nc1]
or a pharmaceutical acceptable salt thereof, to thereby treat or prevent the peripheral neuropathy.

10. The method of claim 9, wherein the peripheral neuropathy is Charcot-Marie Tooth Disease.

11. The method of claim 9, wherein the peripheral neuropathy is chemotherapy-induced peripheral neuropathy.

12. The method of claim 11, wherein the chemotherapy-induced peripheral neuropathy is taxol-induced peripheral neuropathy or vincristine-induced peripheral neuropathy.

13. The method of claim 1, wherein the subject is a human.
